(19) United States
(12) Patent Application Publication
(10) Pub. No.: US 2019/0269656 A1
(43) Pub. Date: Sep. 5, 2019

(54) Combination Therapies Using Indazolylbenzamide Derivatives for the Treatment of Cancer

(71) Applicant: ESANEX, INC., Indianapolis, IN (US)

(72) Inventor: Everardus O.M. ORLEMANS, Chapel Hill, NC (US)

(21) Appl. No.: 16/334,067

(22) PCT Filed: Sep. 25, 2017
(86) PCT No.: PCT/US2017/053266

§ 371 Date: Mar. 18, 2019

Related U.S. Application Data
(60) Provisional application No. 62/399,306, filed on Sep. 23, 2016.

(51) Int. Cl. A61K 31/416; A61P 35/00; A61K 31/4433

(52) U.S. Cl. CPC A61K 31/416; A61K 31/4433; A61P 35/00

(57) ABSTRACT

The invention relates to combination therapies useful in the treatment and/or prevention of cancer.

### CROSS-REFERENCE TO RELATED APPLICATION

This application claims priority to U.S. Provisional Application No. 62/399,306, filed Sep. 23, 2016, the disclosure of which is hereby incorporated by reference in its entirety.

### BACKGROUND OF THE INVENTION

### Field of the Invention

The invention relates to combination therapies useful in the treatment and/or prevention of diseases and/or conditions related to cell proliferation, such as cancer.

### Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell 2 division in normal cells and continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Research, 46, 467-473 (1986), Folkman, J., Journal of the National Cancer Institute, 82, 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al., The New England Journal of Medicine, 324(1), 1-8 (1991). Under conditions of unregulated angiogenesis, therapeutic methods designed to control, repress, and/or inhibit angiogenesis could lead to the abrogation or mitigation of these conditions and diseases.

Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock protein 90 (Hsp90) chaperone proteins stabilize well over 200 different known client proteins helping them to fold correctly as they take up their rightful positions in the cell. Inhibitors of the chaperone protein Hsp90 are of current interest because of the central role of Hsp90 in the maturation and maintenance of numerous proteins that are critical for tumor cell viability and growth. Possible, relevant Hsp90 clients for the tumor types under investigation include mutated STK11/LKB1 (Boudeau j et al. Biochem. J. 370: 849-857 (2003)) and NF1 null (De Raedt T et al., Cancer Cell 20(3):400-13 (2011)) in the NSCLC population, and DNA methyltransferase-1 (Yamaki H et al., J Antibiot (Tokyo) 64(9):635-44 (2011)) in the SCLC population.

Neuroendocrine tumors (NETs) are endocrine neoplasms occurring most frequently in gastrointestinal and broncho-pulmonary (BP) systems. The majority of patients present with advanced disease for which few treatment options exist.

Molecular genetic studies have revealed that the development of NETs may involve different genes, each of which may be associated with several different abnormalities that include point mutations, gene deletions, DNA methylation, chromosomal losses and chromosomal gains. For example, foregut NETs have frequent deletions and mutations of the MEN1 gene, whereas midgut NETs have losses of chromosome 18, 11q and 16q, and hindgut NETs express transforming growth factor-α and the epidermal growth factor receptor. Furthermore, in lung NETs, a loss of chromosome 3p is the most frequent change and p53 mutations and chromosomal loss of 5q21 are associated with more aggressive tumors and poor survival. In addition, methylation frequencies of retinoic acid receptor-β, E-cadherin and RAS-associated domain family genes increase with the severity of lung NETs. Thus, the development and progression of NETs is associated with specific genetic abnormalities that indicate the likely involvement of different molecular pathways. Small intestine neuroendocrine tumors (SINETs) are the most common malignancy of the small bowel. Analysis of 48 SI-NETs by massively parallel exome sequencing detected an average of 0.1 somatic single nucleotide variants (SNVs) per 106 nucleotides (range, 0-0.59). 197 protein-altering somatic SNVs affected a preponderance of cancer genes, including FGFR2, MEN1, HOOK3, EZH2, MLF1, CARD11, VHL, NONO, and SMAD1. Candidate therapeutically relevant alterations were found in 35 patients, including SRC, SMAD family genes, AURKA, EGFR, HSP90, and PDGFR. Most of these affected cancer genes are Hsp90 clients and down-regulated by Hsp90 inhibition (Wu Z et al, Molecular & Cellular Proteomics 11(6): M 111.016675 (2012)).

### BRIEF DESCRIPTION OF THE DRAWINGS

FIG. 1 provides a best tumor response in each patient treated as disclosed in Example 1. SD is stable disease; PD is progressive disease; ORR is overall response rate.

FIG. 2 provides a best tumor response in each patient treated as disclosed in Example 1 that was previously treated with 2 or less prior lines of treatment. SD is stable disease; PD is progressive disease; ORR is overall response rate.

FIG. 3 provides treatment duration by tumor location, grade, proliferation marker Ki67 value, and number of prior lines of treatment.

FIG. 4 provides treatment duration of gastrointestinal (GI), lung, and unknown (Unk) tumors, based on tumor type, ≤2 prior lines of treatment and Ki67<20%. SD is stable disease; PD is progressive disease; ORR is overall response rate.

FIG. 5 shows response in patient with pulmonary carcinoid NET. CT scan shows left iliac adenopathy marked with two diameters in cm. Baseline tumor measured at 6.51 cm and 4.25 cm, and after 4 cycles of treatment tumor measured at 3.75 cm and 2.42 cm.

### SUMMARY OF THE INVENTION

The inventors have discovered that the Hsp90 inhibitors of this disclosure in combination with everolimus are efficient in treatment of cancer.

In one aspect, the disclosure provides methods for treating cancer in a subject in need thereof, the method comprising administering to the subject:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, wherein the Hsp90 inhibitor is administered in an amount of about 50 mg/m² to about 150 mg/m²; and
b) everolimus administered in an amount of about 2 mg to about 20 mg.

In another aspect, the disclosure provides methods for treating cancer in a subject in need thereof, the method comprising administering a therapeutically effective amount of:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof; and
b) everolimus;
to the subject on a dosage schedule, wherein the dosage schedule comprises at least two 28-day treatment cycles, and wherein:
   (i) the Hsp90 inhibitor is administered every other day starting on day 1 and continuing for at least 21 days of each 28-day treatment cycle; and
   (ii) everolimus is administered daily starting on day 1 of each 28-day treatment cycle.
      In one embodiment of this aspect, the Hsp90 inhibitor is administered in an amount of about 50 mg/m² to about 150 mg/m²; and everolimus is administered in an amount of about 2 mg to about 20 mg.

### DETAILED DESCRIPTION OF THE INVENTION

Before the disclosed methods are described, it is to be understood that the aspects described herein are not limited to specific embodiments, or compositions, and as such can, of course, vary. It is also to be understood that the terminology used herein is for the purpose of describing particular aspects only and, unless specifically defined herein, is not intended to be limiting.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The term “pharmaceutical composition” is used in its widest sense, encompassing all pharmaceutically applicable compositions containing at least one active substance, and optional carriers, adjuvants, constituents etc. The term “pharmaceutical composition” also encompasses a composition comprising the active substance in the form of derivative or pro-drug, such as pharmaceutically acceptable salts and esters. The manufacture of pharmaceutical compositions for different routes of administration falls within the capabilities of a person skilled in medicinal chemistry.

In view of the present disclosure, the methods described herein can be configured by the person of ordinary skill in the art to meet the desired need. In general, the disclosed methods provide improvements in the treatment of cancer.

Surprisingly, the inventors have found that the methods of the disclosure were more efficient in treatment of cancer. For example, when subjects (patients) with NETs were treated with the methods of the disclosure, 3 of 17 (18%) patients had partial response rate and 11 of 17 (64%) patients had stable disease as best response. Also, the subjects remained receiving the combination of SNX-5422 plus everolimus for prolonged periods of time, including 2 subjects that are receiving ongoing treatment for over 2 years (i.e., ≥30 cycles). In contrast, when the patients with NETs were treated with everolimus only, without SNX-5422, about 2% patients had partial response rate (Yao C J et al. Everolimus for the treatment of advanced, non-functional neuroendocrine tumours of the lung or gastrointestinal tract (RADIANT-4): a randomised, placebo-controlled, phase 3 study. Lancet 2016; 387: 968-77). Furthermore, the RADIANT-4 study didn't allow enrollment of patients with more than one prior lines of treatment, prior mTor inhibitor use, or poorly differentiated or high grade neuroendocrine tumors. The combination study of SNX-5422 plus everolimus included patients with any differentiation histology of neuroendocrine tumors, prior mTor inhibitor use, and included 11 of 17 patients with two, three, four, five, or even six prior lines of treatment.

The methods of the disclosure are particularly useful in treatment of neuroendocrine cancer, breast cancer, kidney cancer, bladder cancer, head-and-neck cancer, cervical cancer, esophageal cancer, lung cancer, lymphoma, leukemia, multiple myeloma, colon cancer, or liver cancer. In some embodiments, the methods of the disclosure are used in treatment of neuroendocrine cancer. In other embodiments, the methods of the disclosure are used in treatment of neuroendocrine cancer of the stomach and/or intestine (gastrointestinal). In other embodiments, the methods of the disclosure are used in treatment of pancreatic neuroendocrine cancer. In other embodiments, the methods of the disclosure are used in treatment of neuroendocrine cancer of the lung. In some other embodiments, the methods of the disclosure are used in treatment of carcinoids, such as lung, liver, or gastrointestinal.

In some embodiments of this disclosure, the subject in need is a human subject or patient. In some embodiments the subject, e.g., a human, has been previously treated with an anticancer therapy (e.g., surgery, chemotherapy, radiation therapy, hormonal therapy, and Immunotherapy). In some other embodiments the subject has not been previously treated with an anticancer therapy.

The methods of the disclosure require an Hsp90 inhibitor or a pharmaceutically acceptable salt thereof. In some embodiments, the Hsp90 inhibitor is 4-(6,6-dimethyl-4-oxo-3-trifluoromethyl-4,5,6,7-tetrahydro-indazol-1-yl)-2-(trans-4-hydroxy-cyclohexylamino)-benzamide:

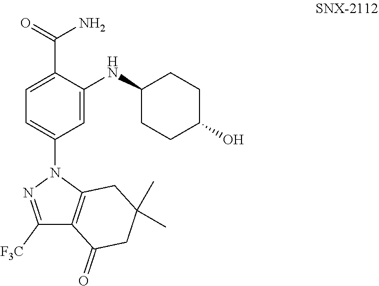 [SMILES: CC1(C)CC(=O)c2c(C(F)(F)F)nn(-c3ccc(C(N)=O)c(N[C@H]4CC[C@H](O)CC4)c3)c2C1]

or a pharmaceutically acceptable salt thereof. Synthesis and characterization data for SNX-2112 is described in U.S. Pat. No. 7,358,370, which is incorporated by reference in its entirety.

In some embodiments, the Hsp90 inhibitor is trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate:

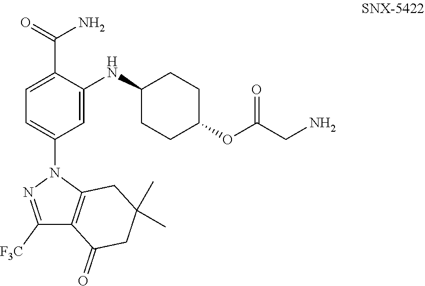 [SMILES: CC1(C)CC(=O)c2c(C(F)(F)F)nn(-c3ccc(C(N)=O)c(N[C@H]4CC[C@H](OC(=O)CN)CC4)c3)c2C1]

or a pharmaceutically acceptable salt thereof. Synthesis and characterization data for SNX-5422 is described in U.S. Pat. No. 7,358,370, which is incorporated by reference in its entirety.

In the methods of the disclosure the Hsp90 inhibitor is administered in an amount of about 50 mg/m² to about 150 mg/m², or about 50 mg/m² to about 100 mg/m², or about 75 mg/m² to about 100 mg/m². In one embodiment, the Hsp90 inhibitor is administered in an amount that gradually increases from about 50 mg/m² to about 100 mg/m². In another embodiment, the Hsp90 inhibitor is administered in an amount of about 100 mg/m². In another embodiment, the Hsp90 inhibitor is administered in an amount of about 75 mg/m². In another embodiment, the Hsp90 inhibitor is administered in an amount of about 50 mg/m².

The methods of the disclosure require everolimus (also known as 42-O-(2-Hydroxyethyl)rapamycin, Afinitor, Certican, or Zortress). Everolimus ((1R,9S,12S,15R,16E,18R,19R,21R,23S,24E,26E,28E,30S,32S,35R)-1,18-dihydroxy-12-{(1R)-2-[(1 S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04,9]hexatriaconta-16,24,26,28-tetraene-2,3,10,14,20-pentaone) has the following chemical structure:

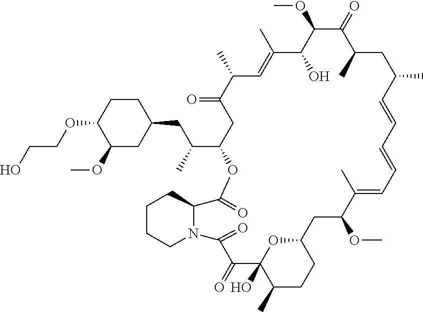 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In the methods of the disclosure, everolimus is administered in an amount of about 5 mg to about 20 mg, or about 7 mg to about 20 mg, or about 8 mg to about 20 mg, or about 10 mg to about 20 mg, or about 2 mg to about 18 mg, or about 2 mg to about 15 mg, or about 2 mg to about 12 mg, or about 2 mg to about 10 mg, or about 5 mg to about 15 mg, or about 7 mg to about 15 mg, or about 8 mg to about 15 mg, or about 10 mg to about 15 mg, or about 5 mg to about 18 mg, or about 5 mg to about 15 mg, or about 5 mg to about 12 mg, or about 5 mg to about 10 mg, or about 8 mg to about 12 mg, or about 9 mg to about 11 mg, or about 9.5 mg to about 10.5 mg, or about 10 mg. In one embodiment, everolimus is administered in an amount of about 10 mg.

In some embodiments, everolimus is administered after the Hsp90 inhibitor is administered. In some embodiments, everolimus is administered at least between 6 hours and 10 hours after the Hsp90 inhibitor is administered. In some embodiments, everolimus is administered at least 6 hours, or at least 8 hours, or at least 10 hours after the Hsp90 inhibitor is administered. In some embodiments, everolimus is administered no more than 16 hours, or no more than 14 hours, or no more than 12 hours after the Hsp90 inhibitor is administered.

In an exemplary, non-limiting embodiment of the methods of the disclosure, the Hsp90 inhibitor is administered in an amount of about 50 mg/m² to about 150 mg/m²; and everolimus is administered in an amount of about 2 mg to about 20 mg. In another exemplary, non-limiting embodiment of the methods of the disclosure, the Hsp90 inhibitor is administered in an amount of about 50 mg/m² to about 100 mg/m²; and everolimus is administered in an amount of about 8 mg to about 12 mg. In another exemplary, non-limiting embodiment of the methods of the disclosure, the Hsp90 inhibitor is administered in an amount of about 50 mg/m² to about 100 mg/m²; and everolimus is administered in an amount of about 10 mg. In another exemplary, non-limiting embodiment of the methods of the disclosure, the Hsp90 inhibitor is administered in an amount of about 75 mg/m²; and everolimus is administered in an amount of about 10 mg.

In some methods of the disclosure, the Hsp90 inhibitor and everolimus may be administered simultaneously, separately, or sequentially in the methods of the disclosure.

In other methods of the disclosure, the Hsp90 inhibitor and everolimus are administered according to the dosage schedule. In one embodiment, the dosage schedule comprises two, or three, or four, or five, or six 28-day treatment cycles, and wherein:
(i) the Hsp90 inhibitor is administered every other day for at least 21 days during each 28-day treatment cycle; and
(ii) everolimus are administered once every day starting on day 1 of the first 28-day treatment cycle.
   In one embodiment, the dosage schedule comprises four 28-day treatment cycles.
   In another embodiment, the dosage schedule comprises six 28-day treatment cycles.

### Pharmaceutical Compositions

In some embodiments, the method comprises the administration of the Hsp90 inhibitor in a pharmaceutical composition having at least one pharmaceutically acceptable carrier, solvent, adjuvant or diluent.

The Hsp90 inhibitor described herein may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. The pharmaceutical compositions described herein may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

### EXAMPLES

The methods of the disclosure are illustrated further by the following examples, which are not to be construed as limiting the invention in scope or spirit to the specific procedures described in them.

### Example 1

Starting on Day 1, SNX-5422 was dosed once every other day for 21 days (11 doses), followed by a 7-day drug free period. SNX-5422 was dosed each morning starting at 50 mg/m² with standard 3+3 dose escalation. The actual dose administered was based on body surface area, calculated using body weight measured at the start of each cycle and height at Screening. The calculated dose was then rounded to the nearest mg on dosing chart. Everolimus was dosed at 10 mg once daily in the evening (at least 8 hours after SNX-5422 dosing) for 28 days, with dose de-escalation allowed based on EVR toxicity. Eligible patients were to have unresectable gastro-entero-pancreatic or pulmonary NETs and less than 5 prior lines of anti-cancer treatment.

Tumor response was assessed at the end of every 2 cycles±2 weeks, using RECIST 1.1 criteria (Eisenhauer et al. European Journal of Cancer 45:228-247 (2009)). Partial Response (PR) was defined as at least a 30% decrease in the sum of diameters of target lesions, taking as reference the baseline sum diameters. Progressive Disease (PD) was defined as at least a 20% increase in the sum of diameters of target lesions, taking as reference the smallest sum on study. Stable disease is noted where there is no sufficient shrinkage to qualify for PR or no sufficient increase to qualify for PD.

Results: Seventeen patients (10 male, 7 female; median age 59 years) with NETs were enrolled in the study. The maximum tolerated dose (MTD) of SNX-5422 was determined to be 75 mg/m² in combination with everolimus.

Tolerability: Most frequent (i.e., occurring in 6 or more patients overall) Treatment-Emergent Adverse Events (TEAEs) are provided in Table 1. All patients reported at least one TEAE. Diarrhea was the most commonly reported TEAE, occurring in 80% of patients overall. In 13 patients (65%), the event was considered related to the combination of SNX-5422 and everolimus. Adverse events associated with everolimus included anemia, Aspartate Aminotransferase (AST) increased, platelet count decreased, and fatigue.

TABLE 1^{a}

| | SNX-5422 | | | | SNX-5422 | | | | Overall | | | |
|---|---|---|---|---|---|---|---|---|---|---|---|---|
| | 50 mg/m²; N = 3 | | | | 75 mg/m²; N = 17 | | | | N = 20 | | | |
| | Number of Patients (%) | | | | | | | | | | | |
| Preferred | All | | | | All | | | | All | | | |
| term | Cause | | Related | | Cause | | Related | | Cause | | Related | |
| Any TEAE | 3 | (100) | 3 | (100) | 17 | (100) | 12 | (71) | 20 | (100) | 15 | (75) |
| Diarrhea | 2 | (67) | 1 | (33) | 14 | (82) | 12 | (71) | 16 | (80) | 13 | (65) |

| Hyponatremia | 1 | (33) | 0 | 9 | (53) | 2 | (12) | 10 | (50) | 2 | (10) |
|---|---|---|---|---|---|---|---|---|---|---|---|
| Anemia | 2 | (67) | 0 | 8 | (48) | 4 | (24) | 10 | (50) | 4 | (20) |
| Vomiting | 0 | | 0 | 8 | (48) | 3 | (18) | 8 | (40) | 3 | (15) |
| Nausea | 1 | (33) | 0 | 6 | (35) | 4 | (24) | 7 | (35) | 4 | (20) |
| Hypokalemia | 0 | | 0 | 6 | (35) | 1 | (6) | 6 | (30) | 1 | (5) |
| AST increased | 0 | | 0 | 6 | (35) | 1 | (6) | 6 | (30) | 1 | (5) |
| Platelet count | 0 | | 0 | 6 | (35) | 2 | (12) | 6 | (30) | 2 | (10) |
| decreased | | | | | | | | | | | |
| Fatigue | 1 | (33) | 0 | 5 | (29) | 4 | (24) | 6 | (30) | 4 | (20) |

| | | | | | | | | |
|---|---|---|---|---|---|---|---|---|
| Weight decreased | 0 | 0 | 6 | (35) | 0 | 6 | (30) | 0 |

Note:
TEAEs associated with everolimus are in italics.

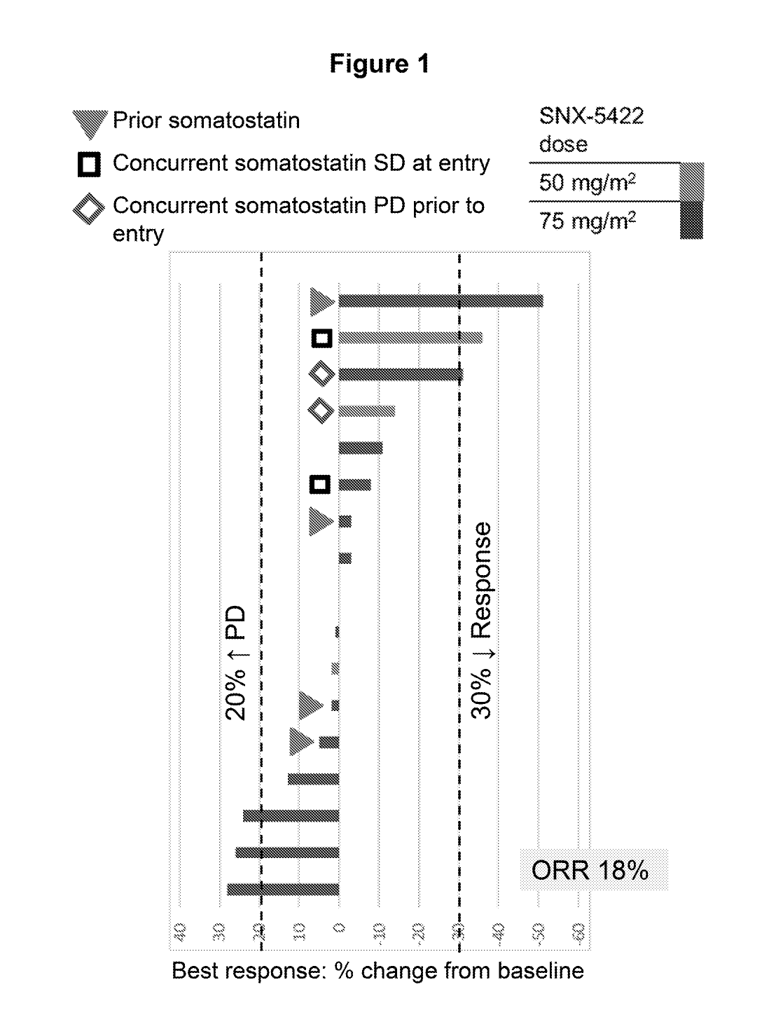

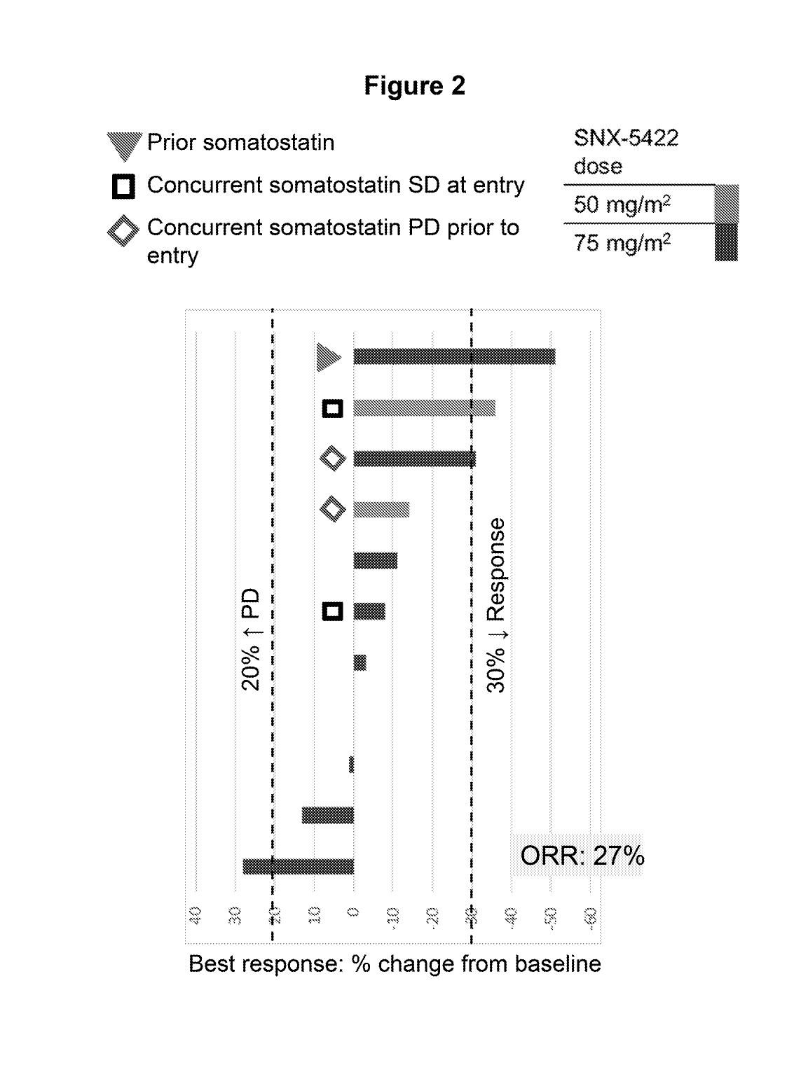

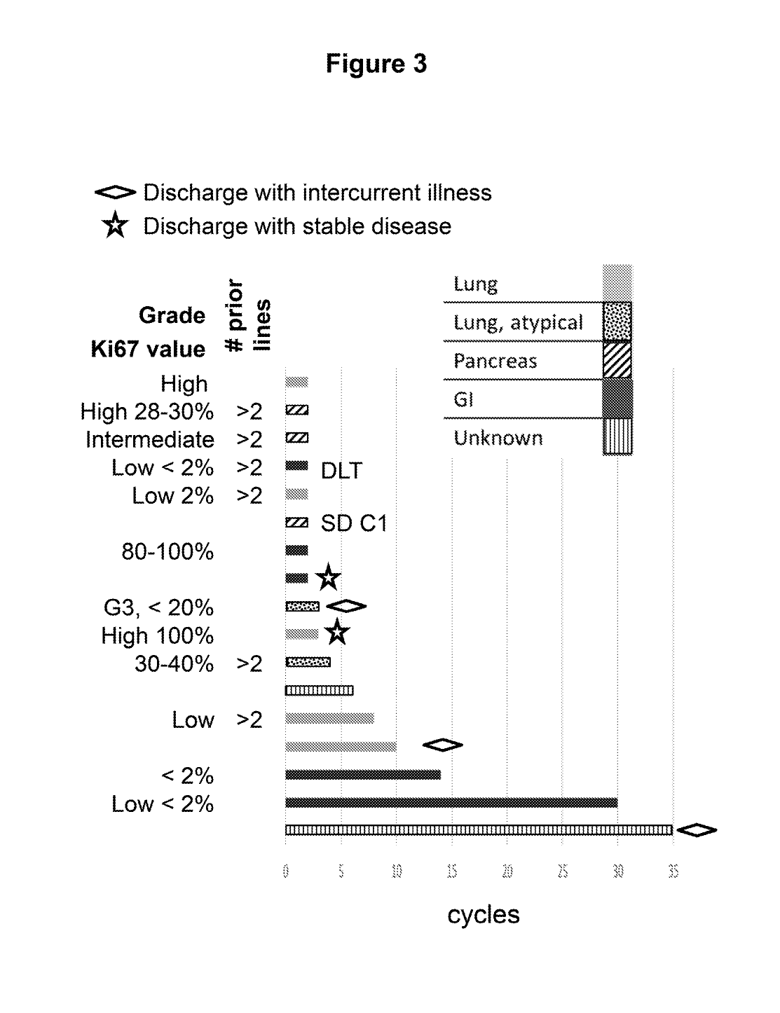

Of 17 NET patients evaluable for efficacy, 3 had partial responses (18%), 11 stable disease (64%), and 3 (18%) progressive disease as best response. Best response results for all 17 patients are illustrated in FIG. 1. Of 12 patients with stable disease, 2 patients have ongoing treatment (one ongoing >26 cycles), 3 patients withdrew from study, and the remaining patients ultimately progressed. One patient has achieved a 51% reduction in target lesion size. Four patients are currently ongoing, and two of these patients have completed over 26 cycles. FIG. 2 provides a best tumor response in each patient previously treated with 2 or less prior lines of treatment. PD is progressive disease; ORR is overall response rate. 3 patients had partial response, 7 patients had stable disease, and 1 patient had progressive disease as best response. As provided in FIG. 3, best response to combination of SNX-5422 and everolimus was observed in patients with <2 prior lines of anti-cancer therapy, and/or those that exhibited Ki67<20%.

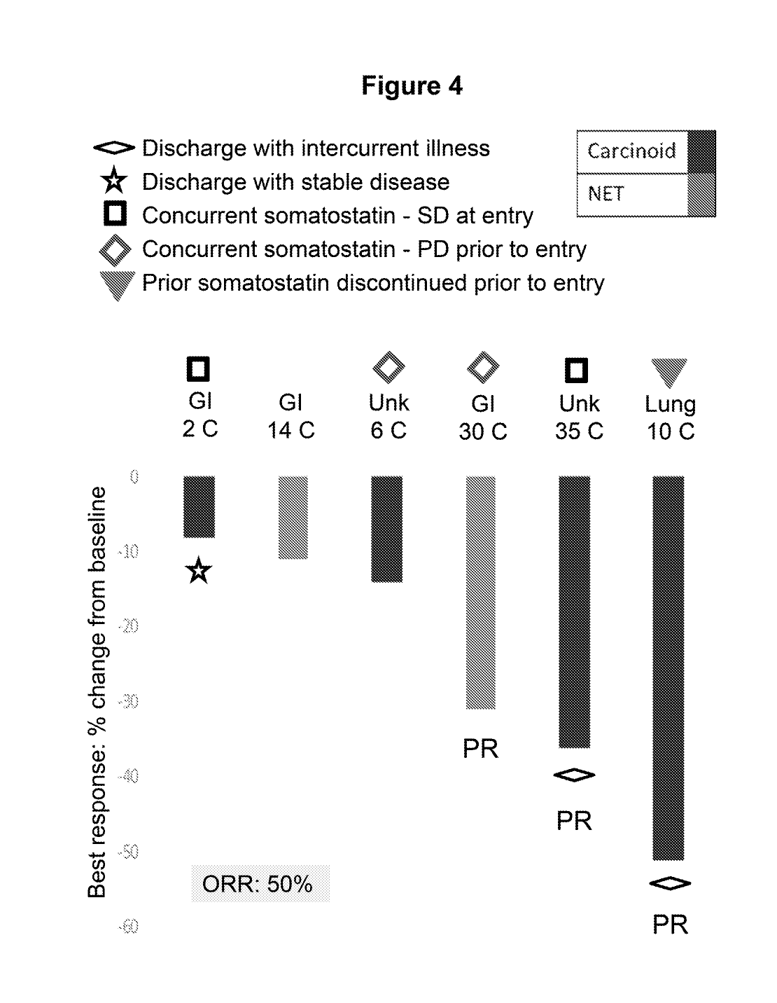

FIG. 4 presents outcomes of the treatment duration for six patients with tumors in the GI tract/lung/unknown carcinoid where the patients had ≤2 prior lines of anti-cancer therapy and also exhibited Ki67<20%. The overall response rate for this group of patients was 50%. As shown in FIG. 4, three patients had partial response. One patient with lung carcinoid received 10 treatment cycles and had 51% tumor reduction; one patient with unknown carcinoid, received 35 treatment cycles and had 36% tumor reduction; and one patient with GI NET received 30 treatment cycles and had 31% tumor reduction.

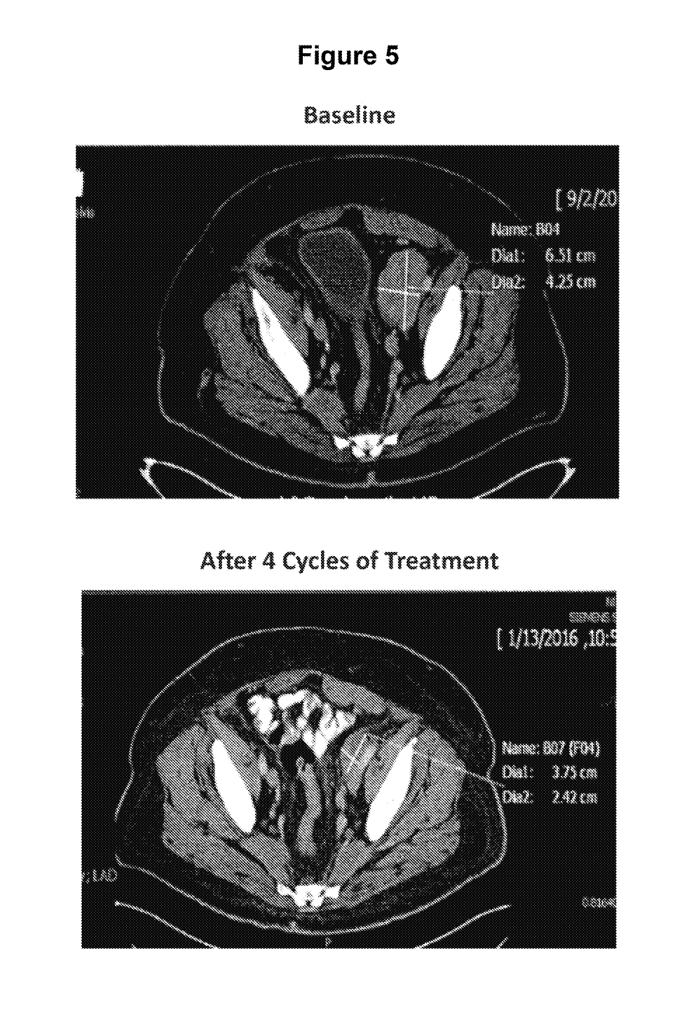

FIG. 5 provides a response in patient with pulmonary carcinoid NET after 4 cycles of treatment (CT scan left iliac adenopathy). Baseline tumor measured at 6.51 cm and 4.25 cm, and decreased to 3.75 cm and 2.42 cm after 4 cycles of treatment. This decrease represents PR. These data show that SNX-5422 in combination with everolimus is useful in treating patients with advanced NETs.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A method for treating cancer in a subject in need thereof, the method comprising administering to the subject:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, wherein the Hsp90 inhibitor is administered in an amount of about 50 mg/m² to about 150 mg/m²; and
b) everolimus administered in an amount of about 2 mg to about 20 mg.

2. A method for treating cancer in a subject in need thereof, the method comprising administering a therapeutically effective amount of:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof; and
b) everolimus;
to the subject on a dosage schedule, wherein the dosage schedule comprises at least two 28-day treatment cycles, and wherein:
(i) the Hsp90 inhibitor is administered every other day starting on day 1 and continuing for at least 21 days of each 28-day treatment cycle; and
(ii) everolimus is administered daily starting on day 1 of each 28-day treatment cycle.

3. A method of claim 2, wherein the Hsp90 inhibitor is administered in an amount of about 50 mg/m² to about 150 mg/m²; and everolimus is administered in an amount of about 2 mg to about 20 mg.

4. A method of claim 1, wherein the Hsp90 inhibitor is trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate.

5. A method of claim 1, wherein the Hsp90 inhibitor is administered in an amount of about 50 mg/m² to about 100 mg/m².

6. A method of claim 1, wherein the Hsp90 inhibitor is administered in an amount that gradually increases from about 50 mg/m² to about 100 mg/m².

7. (canceled)

8. A method of claim 1, wherein everolimus is administered in an amount of about 5 mg to about 20 mg, or about 7 mg to about 20 mg, or about 8 mg to about 20 mg, or about 10 mg to about 20 mg, or about 2 mg to about 18 mg, or about 2 mg to about 15 mg, or about 2 mg to about 12 mg, or about 2 mg to about 10 mg, or about 5 mg to about 15 mg, or about 7 mg to about 15 mg, or about 8 mg to about 15 mg, or about 10 mg to about 15 mg, or about 5 mg to about 18 mg, or about 5 mg to about 15 mg, or about 5 mg to about 12 mg, or about 5 mg to about 10 mg, or about 8 mg to about 12 mg, or about 9 mg to about 11 mg, or about 9.5 mg to about 10.5 mg, or about 10 mg.

9. A method of claim 1, wherein everolimus is administered in an amount of about 10 mg.

10. A method of claim 5, wherein everolimus is administered at least between 6 hours and 10 hours after the Hsp90 inhibitor is administered.

11. A method of claim 5, wherein the dosage schedule comprises three, or four, or five, or six 28-day treatment cycles.

12. A method of claim 1, wherein cancer is neuroendocrine cancer, breast cancer, kidney cancer, bladder cancer, head-and-neck cancer, cervical cancer, esophageal cancer, lung cancer, lymphoma, leukemia, multiple myeloma, colon cancer, or liver cancer.

13. A method of claim 12, wherein neuroendocrine cancer is pancreatic neuroendocrine cancer.

14. A method of claim 12, wherein neuroendocrine cancer is of the stomach and/or intestine (gastrointestinal).

15. A method of claim 12, wherein neuroendocrine cancer is of the lung.

16. A method of claim 12, wherein carcinoid is lung, liver, or gastrointestinal.

17. A method of claim 2, wherein the Hsp90 inhibitor is trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate.

18. A method of claim 17, wherein the Hsp90 inhibitor is administered in an amount of about 50 mg/m² to about 100 mg/m².

19. A method of claim 17, wherein the Hsp90 inhibitor is administered in an amount that gradually increases from about 50 mg/m² to about 100 mg/m².

20. A method of claim 2, wherein cancer is neuroendocrine cancer, breast cancer, kidney cancer, bladder cancer, head-and-neck cancer, cervical cancer, esophageal cancer, lung cancer, lymphoma, leukemia, multiple myeloma, colon cancer, or liver cancer.

21. A method of claim 2, wherein everolimus is administered at least between 6 hours and 10 hours after the Hsp90 inhibitor is administered.

22. A method of claim 2, wherein the dosage schedule comprises three, or four, or five, or six 28-day treatment cycles.
